(19) United States
(12) Patent Application Publication
(10) Pub. No.: US 2023/0182347 A1
(43) Pub. Date: Jun. 15, 2023

(54) HIGH RESOLUTION 3D PRINTING PROCESS OF COMPLEX STRUCTURES

(71) Applicant: 4DEVICES MEDICAL SAGL, Gordola (CH)

(72) Inventor: Carmela DE MARCO, Aarau (CH)

(73) Assignee: 4DEVICES MEDICAL SAGL, Gordola (CH)

(21) Appl. No.: 17/922,916

(22) PCT Filed: May 5, 2021
(86) PCT No.: PCT/IB2021/053788

§ 371 Date: Nov. 2, 2022

(30) Foreign Application Priority Data
May 7, 2020 (CH) 00546/20
May 7, 2020 (IT) 102020000010219

(51) Int. Cl. B29C 33/38; B29C 64/10; B33Y 10/00; B33Y 80/00

(52) U.S. Cl. CPC B29C 33/3842; B29C 64/10; B33Y 10/00; B33Y 80/00; B29L 2031/757

(57) ABSTRACT

A printing process of high resolution, preferably medical, devices with complex geometries is described, comprising the steps of: printing a model (1) with a three-dimensional printing method by using a three-dimensional printer; said model (1) positive reproducing the medical device (10) to be made; - said model (1) being printed of a first water-soluble polymer (2) or aqueous solutions; covering said model (1) with a layer of material (3) insoluble to a solution able to dissolve said first soluble polymer (2); said covering step making a shell of solid mold (7) provided with a surface comprising empty interstitial spots; - infiltrating an amount of water or aqueous solution into said solid mold through said empty interstitial spots so that to dissolve said model (1) and to make a mold cavity (8) negative reproducing said model (1); - infiltrating into the mold

### FIELD OF THE INVENTION

The present invention concerns a three-dimensional printing process of high resolution, preferably medical, devices with complex geometries.

### KNOWN ART

As known, many medical applications in which, for example, an artificial or natural stoma must be held open permanently (non-resorbable) or temporarily (resorbable) in a membrane, wall, etc. require the insertion of a small medical device, such as a stent. An application example is the treatment of hydrocephalus by inserting a loop/stent.

A further use example of very small devices is represented by stents for the treatment of glaucoma, or scaffolds for tissue engineering or even stents used for the anastomoses of small vessels.

Such medical devices should be able to reach very small dimensions, could, for example, be inscribed in cylindrical tubular elements of less than 1 cm in length or of less than 5 mm in diameter.

The Applicant has observed that in the biomedical field, a vast class of biocompatible polymeric materials (polylactic acid (PLA), polyglycolic acid (PGA) and their copolymers such as polylactic-co-glycolic acid (PLGA), polycaprolactone (PCL), polyvinyl alcohol (PVA), thermoplastic polyurethanes (TPU), polyester urethanes (PEU) and polypropylene carbonates (PPC)) are of interest for the implementation of medical devices, such as for example stents, although the current three-dimensional molding techniques are limited to making cylindrical shapes with diameters of no more than 4-5 mm.

The Applicant has observed that for such type of devices, for example in the medical field, not all the materials can be printed with the current three-dimensional or 3D printing techniques. In particular, the thermoplastic polymers are generally printed with the Fused Deposition Modelling (FDM) technique, which however has the drawback of having low printing resolutions.

The current 3D printing techniques are direct techniques, i.e. are based on the stratification of the material (additive manufacturing) and this means that complex structures cannot be created.

It is possible to print some thermosetting materials through three-dimensional printing techniques, such as stereolithography or Continuous Digital Light manufacturing (cDLM).

However, the Applicant has observed that very few thermosetting materials can be printed with such techniques due to the very high density of energy generally required by the thermosetting resins for their curing.

The Applicant has thus addressed the problem of implementing a three-dimensional printing process of high resolution medical devices with complex geometries.

The Applicant has further addressed the problem of implementing a three-dimensional printing process of high resolution, preferably medical, devices with complex geometries which can use different materials with respect to the thermoplastic or thermosetting materials used up to now by the known art.

### SUMMARY OF THE INVENTION

Thus, in a first aspect, the invention concerns a three-dimensional printing process of high resolution, preferably medical, devices with complex geometries, comprising the steps of:
printing a model with a three-dimensional printing method by using a three-dimensional printer; said model positive reproducing the medical device to be made;
said model being printed of a first water-soluble polymer or aqueous solutions;
covering said model with a layer of material insoluble to a solution able to dissolve said first soluble polymer; said covering step making a shell of solid mold provided with a surface comprising empty interstitial spots;
infiltrating an amount of water or aqueous solution into said solid mold through said empty interstitial spots so that to dissolve said model and to make a mold cavity negative reproducing said model;
infiltrating into the mold cavity, through the outer walls of said mold, a second liquid polymer; said material having a melting temperature greater than the melting temperature of said second liquid polymer and/or a difference in the Hildebrand solubility parameter greater than or equal to 2 cal^{½} cm^{-3/2} with respect to that of said second liquid polymer; said step of infiltrating into the mold cavity occurs by depositing the liquid polymer on the outer walls of said mold;
removing the mold by releasing the product obtained and created in said second polymer.

In the context of the present invention, “second liquid polymer” can be understood as either a thermoplastic polymer in solution, as a thermoplastic polymer above its melting temperature Tm, or as a thermosetting prepolymer.

In the aforesaid aspect, the present invention can have at least one of the preferred characteristics described hereunder.

Preferably, the model is printed with a water-soluble polymer selected between thermoplastics or thermosets.

Advantageously, the first soluble polymer comprises at least one among polyvinyl alcohol, dimethylacrylamide (DMA), methacrylic acid (MA) and its esters, methacrylic anhydride (MAA), polyvinylpyrrolidone (PVP), succinic acid (SAA) and its esters or a combination thereof.

Preferably, the model is completely covered with a layer of material of a maximum thickness of 2 cm.

Preferably, the covering material has a melting temperature greater than the melting temperature of said second polymer or said prepolymer mixture.

Conveniently, said covering material is selected among wax, silicone-based elastomers or perfluoropolyethers.

Advantageously, the second polymer or prepolymer mixture is a biocompatible shape-memory polymer.

Preferably, the second biocompatible shape-memory polymer is selected between a one-way biocompatible shape-memory polymer or a two-way biocompatible shape-memory polymer.

Preferably, the second biocompatible shape-memory polymer is Norland Optical Adhesive 63.

Conveniently, in the step of infiltrating into the mold cavity a second liquid polymer through the outer walls of the mold, the infiltration occurs by capillarity.

Preferably, in the step of infiltrating into the mold cavity a second liquid polymer through the outer walls of the mold, the mold and the second liquid polymer deposited thereon are subjected to the action of a vacuum source which creates a vacuum inside the mold cavity.

Conveniently, in the step of infiltrating into the mold cavity a second liquid polymer through the outer walls of the mold, the infiltration occurs by injecting the second liquid polymer into the mold cavity.

Preferably, in the step of removing the mold, the mold is immersed into an organic solvent so that to be dissolved.

Alternatively, in the step of removing the mold, the latter is removed mechanically.

Alternatively, in the step of removing the mold, said mold is subjected to the heat generated by a heat source to melt it.

Further characteristics and advantages of the invention will become clearer in the detailed description of some preferred, but not exclusive, embodiments of a process according to the present invention.

### BRIEF DESCRIPTION OF THE DRAWINGS

Such description will be exposed hereunder with reference to the accompanying drawings provided by way of example only and thus not limiting, in which:

FIG. 1 shows a block diagram of the printing process of high resolution, preferably medical, devices with complex geometries, according to the present invention; and

FIG. 2 is a schematic view of some steps of a printing process of high resolution, preferably medical, devices with complex geometries according to the present invention.

### DETAILED DESCRIPTION OF EMBODIMENTS OF THE INVENTION

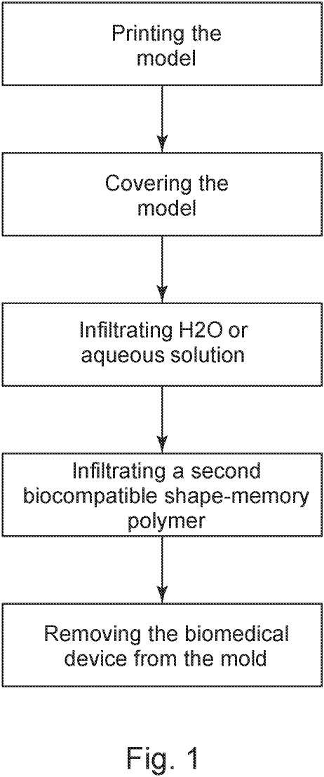

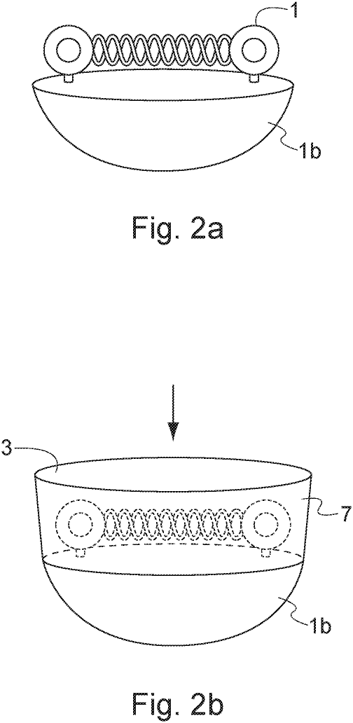

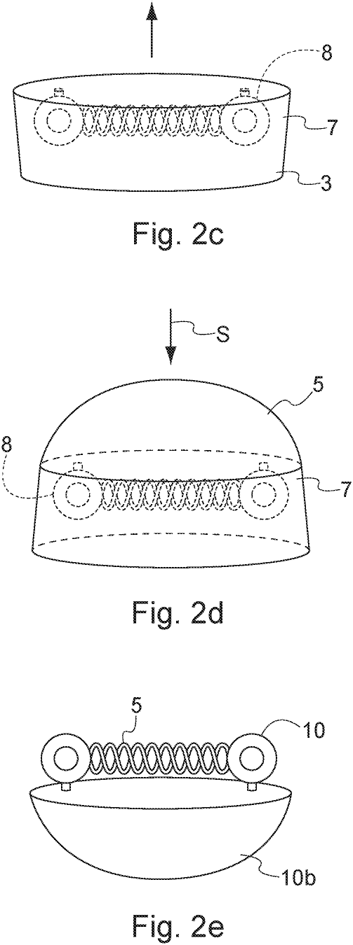

With reference to the figures and in particular to FIGS. 1 and 2, the steps of a printing process of high resolution, preferably medical, devices 10 with complex geometries according to the present invention, are shown.

The process starts with the implementation of a model 1 which positive reproduces the high resolution, preferably medical, device 10 with complex geometries to be made, FIG. 2a.

The model 1 is made by means of three-dimensional printing techniques. Preferably, the model 1 is made by means of three-dimensional printing techniques which use nano- or microscale or milliscale printers.

For example, the model 1 is made with printers that use the Fused Deposition Modelling (FDM) technique or Continuous Digital Light manufacturing (cDLM) and other techniques of stereolithography and molding.

Preferably, the model 1 is made of a first water-soluble polymer 2 or aqueous solutions with a pH of less than 6 or greater than 8. Preferably, in aqueous solutions having a pH of between 3 and 6 or of between 8 and 12. Aqueous solutions adapted for the purpose are basic solutions with a maximum of 1 M NaOH or acidic solutions with a maximum of 1 M HCl.

The model 1 is printed with a water-soluble polymer 2 or aqueous solutions selected between thermoplastics or thermosets.

Preferably, the first soluble polymer 2 comprises at least one among polyvinyl alcohol, dimethylacrylamide (DMA), methacrylic acid (MA) and its esters, methacrylic anhydride (MAA), polyvinylpyrrolidone (PVP), succinic acid (SAA) and its esters or a combination thereof.

The model 1 is printed together with a support 1b which, in the embodiment shown in FIGS. 2a-2b, appears hemispherical in shape with the flat base facing upwards, i.e. facing the model 1 so that to provide a surface for containing the covering material 3.

Whenever the model 1 is printed with a thermoplastic polymer, once the model 1 has been printed it is left to cool, and generally a few minutes are sufficient.

Alternatively, whenever a thermosetting polymer has been printed with stereolithography or with Continuous Digital Light Manufacturing (cDLM) techniques, the model 1 is subjected to a source of UV rays for at least 5 minutes or to a source of heat.

Subsequently, it is thus possible to proceed to a step of covering the model 1 with a layer of material 3 insoluble to a solution able to dissolve the first soluble polymer 2.

The covering step, shown in FIG. 2b, concerns the whole outer surface of the model 1 and allows to make a shell of solid mold 7 provided with an outer surface comprising empty interstitial spots.

In the embodiment shown in FIGS. 1, 2a-2e, the covering material 3 forms a solid mold 7 consisting of a cylindrical body around the model 1, delimited by the support 1b at the bottom.

In order to make the covering step, a layer of material 3 of at least 1 mm thickness, preferably of at least 2 mm, anyhow a layer of substance 3 equal to maximum 2 cm thickness, is deposited over the entire outer surface of the model.

In order for the solid mold 7 to withstand the subsequent infiltration step, the substance 3, which substantially forms the outer walls of the mold, must have a melting temperature greater than the melting temperature of the second liquid, preferably biocompatible, polymer 5 which will subsequently be infiltrated so that to make the final product, i.e. the medical device 10.

Alternatively, always for such purpose, the substance 3, which substantially forms the outer walls of the mold, must have a difference in the Hildebrand solubility parameter greater than or equal to 2 cal^{½}cm^{-3/2} with respect to that of the second liquid polymer 5.

In the embodiment shown, in particular in FIGS. 2b-2d, the covering material 3 has a melting temperature greater than the melting temperature of the second liquid polymer 5 which will subsequently be infiltrated so that to make the final product.

Preferably, the material 3 is selected among wax, silicone-based elastomers or perfluoropolyethers.

Preferably, in the embodiment shown in FIGS. 2b-2d, the material 3 is wax.

Once the mold 7 has been made, as shown in FIGS. 2b-2d, it is possible to immerse the solid mold in water or in a weakly acidic or basic aqueous solution so that the water or solution crosses the empty interstitial spots of the surface of the mold 7 and penetrates therein, thus dissolving the model 1.

By way of example, basic solution is, for example, understood as a solution of 1 M NaOH.

Acidic solution is, for example, understood as a solution of 1 M HCl maximum.

By dissolving the model 1, a mold cavity 8 negative reproducing the model 1 is thus made and consequently, always negative, the medical device 10 to be made, FIG. 2c.

The support 1b made of the same material and integral with the model 1 is also dissolved together with the model 1.

Thus, a second liquid polymer 5 is infiltrated into the mold cavity 8 through the outer walls of the mold 7.

In the present description, “second liquid polymer” can be understood as either a thermoplastic polymer in solution, as a thermoplastic polymer above its melting temperature Tm, or as a thermosetting polymer.

Preferably, the second polymer 5 is a biocompatible polymer.

In the embodiment shown in FIGS. 2d-2e, the second polymer 5 is a biocompatible shape-memory polymer and is at the liquid state when being infiltrated.

Preferably, the second biocompatible shape-memory polymer 5 is selected between a one-way biocompatible shape-memory polymer, generally known as “one-way” in literature, or a two-way biocompatible shape-memory polymer, generally known as “two-way” in literature.

In the embodiment shown in FIG. 2, the second biocompatible shape-memory polymer comprises Norland Optical Adhesive 63.

In the embodiment shown in FIG. 2, the second biocompatible shape-memory polymer is Norland Optical Adhesive 63.

To infiltrate into the mold cavity 8 the second liquid polymer 5, the latter is deposited onto the outer walls of the mold 7.

Whenever the angle of contact between the liquid polymer 5 and the empty interstitial spot is less than 90°, the polymer enters the mold cavity 8 through the interstitial spot by simple capillarity.

Alternatively, whenever the angle of contact between the liquid polymer 5 and the empty interstitial spot is greater than or equal to 90°, an injection system is used for the infiltration or the mold 7 and the second liquid polymer 5 deposited thereon are subjected to the action of a vacuum generator which creates a depression inside the mold cavity 8 to attract the second liquid polymer 5 therein.

Whenever the second biocompatible polymer 5 is a thermoplastic polymer, it is possible to remove the mold thus releasing the product thus obtained and created in the second biocompatible polymer 5 once the second biocompatible polymer 5 is completely infiltrated into the mold cavity and left to polymerize, typically at room temperature and for a period of no less than 20 minutes.

Alternatively, whenever the second biocompatible polymer 5 is a thermosetting polymer, it is necessary to subject it to UV irradiation.

In the embodiment shown in FIG. 2, the preferably medical device 10 is thus made in Norland Optical Adhesive 63.

The step of removing the mold, FIG. 2e, can occur by breaking the mold 7 and by extracting the finished device 10, preferably medical, for example typically when the mold 7 is an elastomer.

In fact, in this case, the mold 7 can be delicately cut and peeled off since the softness of the material it is made of allows its removal without compromising the final object (finished device 10) and its components.

Alternatively, the step of removing the mold 7 can occur by dissolving the latter, i.e. by immersing the mold 7 infiltrated by the second liquid polymer 5 into an organic solvent which will dissolve the mold 7 without affecting the polymer 5.

Preferably, the organic solvent has a difference in the Hildebrand solubility parameter of an absolute value of less than 2 cal^{½}cm^{-3/2} with respect to that of said second liquid polymer 5 and a difference in the Hildebrand solubility parameter of an absolute value greater than 2 cal^{½}cm^{-3/2} with respect to that of said mold 7.

Or, still alternatively, by subjecting the mold 7 to heat, for example by placing the mold 7 in an appropriate furnace.

The temperature to which the furnace should be set and the time of residence therein depend on the material 3 with which the mold 7 is made.

Whenever the polymer 5 is a thermoplastic, the temperature of the furnace must be lower than the melting temperature of the polymer 5, whenever the polymer 5 is a thermoset, the temperature of the furnace must instead be lower than the softening temperature of the thermoset.

The medical device obtained is shown in FIG. 2e constrained to a support 10b made of the same shape-memory polymer 5 as that of the device 10.

The support 10b is subsequently removed.

Several changes can be made to the embodiments described in detail, all anyhow remaining within the protection scope of the invention, defined by the following claims.

1. Process of printing high resolution, preferably medical, devices (10) with complex geometries, comprising the steps of:
printing a model (1) with a three-dimensional printing method by using a three-dimensional printer; said model (1) positive reproducing the medical device (10) to be made;
said model (1) being printed of a first water-soluble polymer (2) or aqueous solutions;
covering said model (1) with a layer of material (3) insoluble to a solution able to dissolve said first soluble polymer (2); said covering step making a shell of solid mold (7) provided with a surface comprising empty interstitial spots;
infiltrating an amount of water or aqueous solution into said solid mold through said empty interstitial spots so that to dissolve said model (1) and to make a mold cavity (8) negative reproducing said model (1);
infiltrating into the mold cavity (8), through the outer walls of said mold, a second liquid polymer (5); said material (3) having a melting temperature greater than the melting temperature of said second liquid polymer (5) and/or a difference in the Hildebrand solubility parameter greater than or equal to 2 cal^{½}cm^{-3/2} with respect to that of said second liquid polymer (5); said step of infiltrating into the mold cavity (8) occurs by depositing the liquid polymer (5) on the outer walls of said mold (7);
removing said mold by releasing the product obtained and created in said second polymer (5).

2. Printing process according to claim 1, characterized in that said model (1) is printed with a soluble polymer (2) selected between thermoplastics or thermosettings.

3. Printing process according to claim 1, characterized in that said first soluble polymer (2) comprises one among polyvinyl alcohol, dimethylacrylamide (DMA), methacrylic acid (MA) and its esters, methacrylic anhydride (MAA), polyvinylpyrrolidone (PVP), succinic acid (SAA) and its esters or a combination thereof.

4. Printing process according to claim 1, characterized in that said model (1) is completely covered with a layer of said material (3) of a maximum of 2 cm in thickness.

5. Printing process according to claim 1, characterized in that said material (3) has a melting temperature greater than the melting temperature of said second liquid polymer (5).

6. Printing process according to any one of claims 1 to 5, characterized in that said material (3) is selected among wax, silicone-based elastomers or perfluoropolyethers.

7. Printing process according to any one of preceding claims 1 to 6, characterized in that said second liquid polymer (5) is a biocompatible shape-memory polymer.

8. Printing process according to claim 7, characterized in that said second biocompatible shape-memory polymer is selected between a one-way biocompatible shape-memory polymer or a two-way biocompatible shape-memory polymer.

9. Printing process according to claim 7, characterized in that said second biocompatible shape-memory polymer is Norland Optical Adhesive 63.

10. Printing process according to claim 1, characterized in that, in said step of infiltrating into the mold cavity (8) a second liquid polymer (5) through the outer walls of said mold (7), the infiltration occurs by capillarity.

11. Printing process according to any one of preceding claims 1 to 10, characterized in that in said step of infiltrating into the mold cavity (8) a second liquid polymer (5) through the outer walls of said mold (7), the mold (7) and said second liquid polymer (5) deposited thereon are subjected to the action of a vacuum source which creates a vacuum inside the mold cavity (8).

12. Printing process according to claim 13, characterized in that in said step of infiltrating into the mold cavity (8) a second liquid polymer (5) through the outer walls of said mold (7), the infiltration occurs by injecting said second liquid polymer (5) into said mold cavity (8).

13. Printing process according to any one of preceding claims 1 to 12, characterized in that, in said step of removing said mold (7), said mold (7) is immersed into an organic solvent so that to be dissolved.

14. Printing process according to any one of preceding claims 1 to 12, characterized in that, in said step of removing said mold (7), said mold (7) is removed mechanically.

15. Printing process according to any one of preceding claims 1 to 12, characterized in that, in said step of removing said mold (7), said mold (7) is subjected to the heat generated by a heat source.
